(19) United States
(12) Patent Application Publication
(10) Pub. No.: US 2009/0093734 A1
(43) Pub. Date: Apr. 9, 2009

(54) METHOD OF INJECTING CARBON DIOXIDE INTO A VASCULAR STRUCTURE AND SYSTEM FOR PRACTISING THE METHOD

(76) Inventor: Carsten Stevenson, Amherstview (CA)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 12/209,230

(22) Filed: Sep. 12, 2008

Related U.S. Application Data
(60) Provisional application No. 60/972,883, filed on Sep. 17, 2007.

(30) Foreign Application Priority Data
Sep. 13, 2007 (CA) 2,602,107

(51) Int. Cl. A61B 5/00

(52) U.S. Cl. 600/560

(57) ABSTRACT

Disclosed is an apparatus comprising: a hose extending, in use, from an automated syringe to a free end; an angiographic catheter; a syringe; and valve having a first port coupled to the catheter, a second port coupled with the syringe and a third port coupled hose free end, the valve being manipulable to connect any two of its ports to one another for communication such that, in use, when the syringe is evacuated, the automated syringe and the apparatus up to the catheter are charged with CO₂ and the catheter is inserted into the vascular system of a patient, the valve (i) can be manipulated to connect the syringe to the catheter such that withdrawal of the plunger of the syringe purges the catheter and (ii) thereafter can be manipulated to connect the catheter to the hose such that evacuation of the automated syringe injects CO₂ into said vascular system.

### CROSS REFERENCE TO RELATED APPLICATION AND PRIORITY CLAIM

This application claims benefit of the filing date of and right of priority of Canadian Patent Application Serial No. 2,602,107 filed Sep. 13, 2007 and U.S. Provisional Patent Application Ser. No. 60/972,883 filed Sep. 17, 2007 under 35 USC § 119(e).

### FIELD OF THE INVENTION

The invention relates to the injection of carbon dioxide into a vascular structure.

### BACKGROUND OF THE INVENTION

Carbon dioxide has been used for decades as an augmentation and/or replacement for typical iodinated contrast media. It is advantageous in the context of patients with renal impairment wherein iodinated contrast media is contra-indicated because of its renal toxicity. Carbon dioxide is also used where large contrast volumes are to be used and iodinated contrast media is contra-indicated because of toxic effects to the patient.

### SUMMARY OF THE INVENTION

The use of an automated syringe to deliver carbon dioxide into the vascular system of a patient forms one aspect of the invention.

Apparatus for use with an automated syringe and a source of carbon dioxide forms another aspect of the invention. This apparatus comprises: a first hose operatively extending, in use, from and in fluid communication with, said automated syringe, to a free end; an angiographic catheter; a syringe; and a first three way valve. The first valve has a first port coupled for fluid communication with the angiographic catheter, a second port coupled for fluid communication with the syringe and a third port coupled to and in fluid communication with the free end of the first hose. The first valve is manipulable to connect any two of its ports to one another for fluid communication such that, in use, when the syringe is evacuated, the automated syringe and the apparatus up to the catheter are charged with carbon dioxide and the catheter is inserted into the vascular system of a patient, the three way valve (i) can be manipulated to connect the syringe to the catheter for fluid communication such that withdrawal of the plunger of the syringe purges the catheter and (ii) thereafter can be manipulated to connect the catheter to the first hose for fluid communication such that evacuation of the automated syringe injects carbon dioxide into said vascular system.

According to another aspect of the invention, the apparatus can further comprise a second three way valve having a first port coupled for fluid communication with the source of carbon dioxide in use, a second port coupled for fluid communication with the automated syringe in use and a third port from which the first hose extends in fluid communication, the second valve being manipulable to fluidly connect any two of its ports to one another.

According to another aspect of the invention, the apparatus can further comprise a second hose coupling, in use, the source of carbon dioxide to the first port of the second valve for fluid communication.

According to another aspect of the invention, the apparatus can further comprise a filter adapted to remove particulate contaminants from the carbon dioxide upstream of the catheter.

A method for delivering carbon dioxide into the vascular system of a patient forms another aspect of the invention. The method comprises the steps of:

(i) providing apparatus according to aspects of the invention;

(ii) coupling the second hose to a source of carbon dioxide for fluid communication;

(iii) evacuating the syringe and purging the apparatus, up to the catheter, of air;

(iv) inserting the catheter into the vascular system of the patient and purging the catheter using the syringe; and

(v) actuating the automated syringe to inject carbon dioxide into said vascular system.

According to another aspect of the invention, in the method, carbon dioxide can be introduced into the second hose in a manner such that pressure in the second hose is maintained above a predetermined minimum pressure at all times.

According to another aspect, the predetermined minimum pressure can be atmospheric pressure.

According to another aspect, in the apparatus provided in step (i), the first hose can be releasably connected to the second valve and the method can further comprise the steps of:

(vi) releasing the first hose from the second valve and removing the catheter from the patient;

(vii) releasably securing the hose of apparatus according to claim 2 to the second valve; and

repeating steps (iii)-(v).

A system for use with an automated syringe and a source of carbon dioxide forms another aspect of the invention. The system comprises: apparatus according to aspects of the invention; and a controller unit coupled in use between and in fluid communication with each of the source of carbon dioxide and the apparatus and adapted to provide an indication if carbon dioxide pressure from the source falls below a predetermined minimum pressure.

According to another aspect, the predetermined minimum pressure can be atmospheric pressure.

According to another aspect, the controller unit can be adapted to deliver carbon dioxide into the apparatus at a selectively variable rate.

According to another aspect, the controller unit can be adapted to provide a readout of source pressure.

According to another aspect, the controller unit can be adapted to provide a readout of pressure in the apparatus.

According to another aspect, the indication can be an audible alarm.

According to another aspect, the apparatus can be disposable and the second hose can be releasably coupled in use to the controller.

The controller itself forms another aspect of the invention.

Use of the system in carrying out the method forms another aspect of the invention.

Use of an angio pump to deliver carbon dioxide into the vascular system of a patient forms another aspect of the invention.

Apparatus for use with an angio pump and a source of carbon dioxide forms yet a further aspect of the invention. This apparatus comprises: a first hose operatively extending, in use, from and in fluid communication with, said angio pump, to a free end; an angiographic catheter; a syringe; and a first three way valve having a first port coupled for fluid communication with the angiographic catheter, a second port coupled for fluid communication with the syringe and a third port coupled to and in fluid communication with the free end of the first hose, the first valve being manipulable to connect any two of its ports to one another for fluid communication such that, in use, when the syringe is evacuated, the angio pump and the apparatus up to the catheter are charged with carbon dioxide and the catheter is inserted into the vascular system of a patient, the three way valve (i) can be manipulated to connect the syringe to the catheter for fluid communication such that withdrawal of the plunger of the syringe purges the catheter and (ii) thereafter can be manipulated to connect the catheter to the first hose for fluid communication such that evacuation of the angio pump injects carbon dioxide into said vascular system.

Other advantages, features and characteristics of the present invention will become more apparent upon consideration of the following detailed description and the appended claims with reference to the accompanying drawings, the latter being briefly described hereinbelow.

### BRIEF DESCRIPTION OF THE FIGURES

FIG. 1 is a schematic view of a system according to an exemplary embodiment of the invention, in use; and

FIG. 2 is a schematic view of a system according to a further exemplary embodiment of the invention, in use.

### DETAILED DESCRIPTION

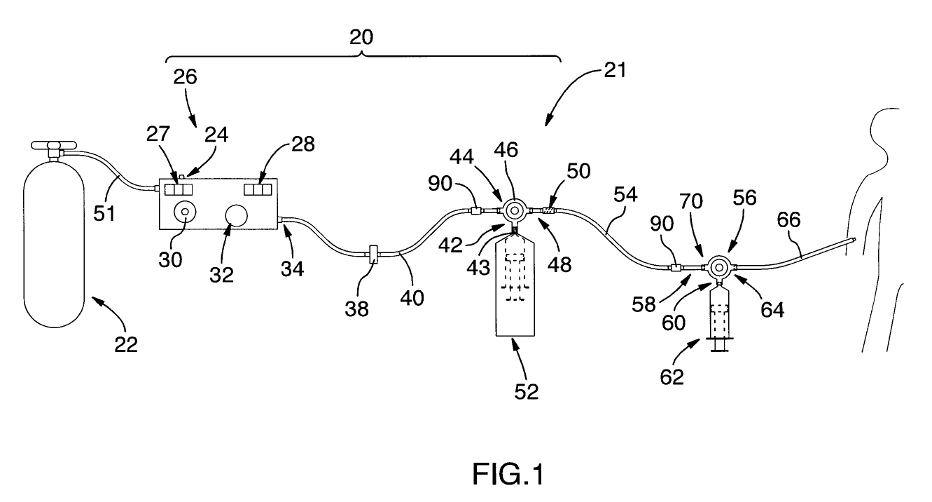

In FIG. 1, an exemplary embodiment of the invention is illustrated in schematic form in use with a source 22 of carbon dioxide, specifically, a tank of carbon dioxide and an automated syringe 52, specifically, an angio pump, which source and pump, for greater certainty, form no part of the invention.

The exemplary embodiment, designated with general reference numeral 20, will be seen to be composed of a disposable hose assembly 21 and a controller unit 26.

The disposable 21 includes a first hose 54, a catheter 66, a syringe 62, a first three way valve 56, a second three way valve 46 and a second hose 40.

The first hose 54 operatively extends from and in fluid communication with, said automated syringe 52, to a free end 70.

The catheter 66 is an angiographic catheter.

The syringe 62 is sized to contain, when fully retracted, 30 cc of fluid.

The first valve 56 has a first port 64 coupled for fluid communication with the angiographic catheter 66, a second port 60 coupled for fluid communication with the syringe 62 and a third port 58 coupled to and in fluid communication with the free end 70 of the first hose 54 via a check valve 90, the first valve 46 being manipulable to connect any two of its ports 64, 58, 60 to one another for fluid communication.

The second three way valve 46 has a first port 44 operatively coupled, via a check valve 90, by the second hose 40 for fluid communication with the source 22 of carbon dioxide in use, a second port 42 coupled by a Luer lock 43 for fluid communication with the automated syringe 52 in use and a third port 48 from which the first hose 54 extends in fluid communication, the third port 48 and the first hose 54 being releasably coupled to one another by a Luer lock 50. The second valve 46 is manipulable to fluidly connect any two of its ports 42, 44, 48 to one another.

A filter 38 is disposed at an intermediate position in the second hose 40 and is adapted to remove particulate contaminants from the carbon dioxide stream.

The controller unit 26 is coupled by a Luer lock 34 to the second hose 40 and to the source of carbon dioxide 22 by a supply hose 51 and is adapted to receive carbon dioxide from the source 22 and deliver said carbon dioxide into the apparatus 21 at a variable rate, selected through the use of a control knob 30. A digital display 27 provides a readout of source pressure, and a further digital display 28 provides a readout of output pressure (pressure in the apparatus). The controller unit 26 is further adapted to emit, through a speaker 32 and a lamp 24, an audible and visual alarm in the event that pressure in the apparatus falls below a predetermined minimum pressure, specifically, atmospheric pressure.

The system 20 is used to advantage in the delivery of carbon dioxide into the vascular system of a patient, shown representatively by patient 68.

In an exemplary embodiment, a methodology of using the apparatus 20 as hereinbefore described commences with the purging of the apparatus, which can be conveniently done, through manipulation of the valves 46, 56, up to the catheter 66. The manner in which such purging can be carried out is largely a matter of routine to persons of ordinary skill in the art, and as such, is not described herein in detail. It is noted, however, that use of the controller 26 permits the operator to ensure that positive carbon pressure is maintained at all times in the apparatus, to minimize the potential for air infiltration; the operator merely needs to monitor pressure and adjust flow accordingly. The check valves 90 also assist in this aspect, as they are arranged to permit carbon dioxide to flow towards the patient and not allow any back flow or air.

Once the catheter has been purged to the satisfaction of the operator, the catheter is inserted into a patient, such as representative patient 68, in a conventional manner as desired by the vascular technician.

Using the syringe 62, the catheter itself 66 is then purged through manipulation of valves 46, 56, again in a manner such that that positive carbon dioxide pressure is maintained at all times in the apparatus.

Once the catheter 66 has been purged, the valves 46, 56 can be manipulated to connect the catheter 66 to the first hose 54 for fluid communication, and to connect the automated syringe 52 to the first hose 54. Once so connected, the automated syringe 52 can be actuated to evacuate the automated syringe 52 and thereby inject carbon dioxide into the vascular system of the subject 68 in a controlled manner. After the injection, the assembly 21 can be removed from the patient and the controller 26 and disposed of, and a new, sterile assembly replaced for use in the next procedure (after purging as previously described).

Persons of ordinary skill in the art will readily appreciate the advantages associated with the system and the method.

One advantage flowing from the use of the angio pump 52 for the delivery of carbon dioxide resides in the precise manner in which carbon dioxide can be injected, in contradistinction to the more irregular flow associated with manual syringe injection. Additionally, angio pumps can have relatively large volumes, which volumes are often desirable in the context of vascular studies.

Another advantage flows from fact that angio pumps are commonly found in renal units; where such pumps are already available, the system can be implemented at relatively low cost.

A further advantage is associated with the positive carbon dioxide pressure which can be maintained in the system at all times, which minimizes the potential for air infiltration, which can be exceedingly dangerous to patients.

Whereas but a single embodiment of the system and a single methodology have been herein described, it should be understood that variations are possible.

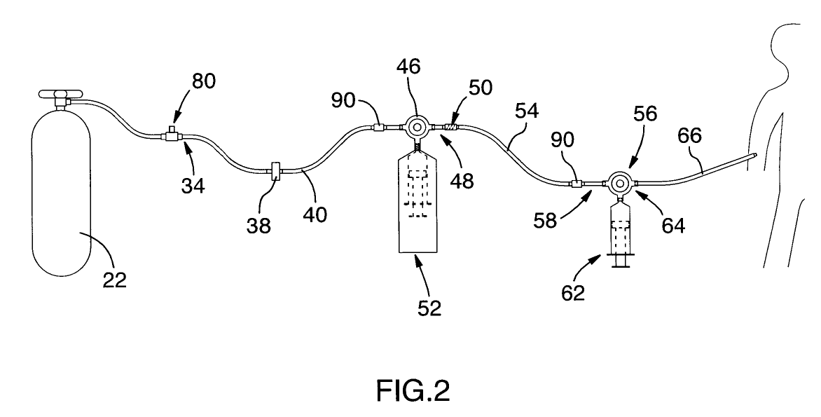

For example, only, FIG. 2 shows an exemplary embodiment wherein the controller 26 is absent, and instead, a pressure relief valve/whistle 80 is provided, and which is coupled to the second valve 46. This embodiment can provide advantages similar to those obtained from the embodiment of FIG. 1. Whereas the rate of carbon dioxide was monitored and controlled in the FIG. 1 embodiment so as to ensure positive pressure is maintained, in this embodiment, the operator is assured that positive pressure is maintained at all times because of the audible whistle associated with gas escaping through the pressure release valve. Of course, use of the system of FIG. 2 would have substantial costs in terms of carbon dioxide utilization (waste), and would not normally be employed, but the example is nonetheless believed useful, for the purpose of understanding the contemplated scope of the claimed invention.

As yet another variation, this time associated with the structure of FIG. 1, after the injection has been completed, only that portion of the structure extending from the catheter to Luer lock 56 can be removed and substituted.

This variation has some benefit, in terms of reduced costs associated with the reuse of, inter alia, hose 40, and simplified purging (since hose 40, first valve 46 and automates syringe 52 do not need to be repurged). However, reuse in this manner may not be permitted in all jurisdictions, and would normally only be permitted in the context of repeated procedures on the same patient in any event.

Further, whereas the invention is described as useful with a specific brand and model of automated syringe, it should be emphasized that the invention can and will be employed with different automated syringes.

In view of the foregoing, the invention should be understood as limited only by the accompanying claims, purposively construed.

1. Use of an automated syringe to deliver carbon dioxide into the vascular system of a patient.

2. Apparatus for use with an automated syringe and a source of carbon dioxide, said apparatus comprising:
a first hose operatively extending, in use, from and in fluid communication with, said automated syringe, to a free end;
an angiographic catheter;
a syringe; and
a first three way valve having a first port coupled for fluid communication with the angiographic catheter, a second port coupled for fluid communication with the syringe and a third port coupled to and in fluid communication with the free end of the first hose, the first valve being manipulable to connect any two of its ports to one another for fluid communication such that, in use, when the syringe is evacuated, the automated syringe and the apparatus up to the catheter are charged with carbon dioxide and the catheter is inserted into the vascular system of a patient, the three way valve (i) can be manipulated to connect the syringe to the catheter for fluid communication such that withdrawal of the plunger of the syringe purges the catheter and (ii) thereafter can be manipulated to connect the catheter to the first hose for fluid communication such that evacuation of the automated syringe injects carbon dioxide into said vascular system.

3. Apparatus according to claim 2, further comprising a second three way valve having a first port coupled for fluid communication with the source of carbon dioxide in use, a second port coupled for fluid communication with the automated syringe in use and a third port from which the first hose extends in fluid communication, the second valve being manipulable to fluidly connect any two of its ports to one another.

4. Apparatus according to claim 3, further comprising a second hose coupling, in use, the source of carbon dioxide to the first port of the second valve for fluid communication.

5. Apparatus according to claim 4, further comprising a filter adapted to remove particulate contaminants from the carbon dioxide upstream of the catheter.

6. A method for delivering carbon dioxide into the vascular system of a patient, the method comprising the steps of:
(i) providing apparatus according to claim 5
(ii) coupling the second hose to a source of carbon dioxide for fluid communication;
(iii) evacuating the syringe and purging the apparatus, up to the catheter, of air;
(iv) inserting the catheter into the vascular system of the patient and purging the catheter using the syringe; and
(v) actuating the automated syringe to inject carbon dioxide into said vascular system.

7. A method according to claim 6, wherein carbon dioxide is introduced into the second hose in a manner such that pressure in the second hose is maintained above a predetermined minimum pressure at all times.

8. A method according to claim 7, wherein the predetermined minimum pressure is atmospheric pressure.

9. A method according to claim 7, wherein in the apparatus provided in step (i), the first hose is releasably connected to the second valve and further comprising the steps of:
(vi) releasing the first hose from the second valve and removing the catheter from the patient;
(vii) releasably securing the hose of apparatus according to claim 2 to the second valve; and
repeating steps (iii)-(v).

10. A system for use with an automated syringe and a source of carbon dioxide, said system comprising:
apparatus according to claim 5; and
a controller unit coupled in use between and in fluid communication with each of the source of carbon dioxide and the apparatus and adapted to provide an indication if carbon dioxide pressure from the source falls below a predetermined minimum pressure.

11. A system according to claim 10, wherein the predetermined minimum pressure is atmospheric pressure.

12. A system according to claim 10, wherein the controller unit is adapted to deliver carbon dioxide into the apparatus at a selectively variable rate.

13. A system according to claim 10, wherein the controller unit is adapted to provide a readout of source pressure.

14. A system according to claim 10, wherein the controller unit is adapted to provide a readout of pressure in the apparatus.

15. A system according to claim 10, wherein the indication is an audible alarm.

16. A system according to claim 10, wherein the apparatus is disposable and the second hose is releasably coupled in use to the controller.

17. A controller according to claim 10.

18. Use of the system according to claim 10.

19. Use of an angio pump to deliver carbon dioxide into the vascular system of a patient.

20. Apparatus for use with an angio pump and a source of carbon dioxide, said apparatus comprising:
a first hose operatively extending, in use, from and in fluid communication with, said angio pump, to a free end;
an angiographic catheter;
a syringe; and
a first three way valve having a first port coupled for fluid communication with the angiographic catheter, a second port coupled for fluid communication with the syringe and a third port coupled to and in fluid communication with the free end of the first hose, the first valve being manipulable to connect any two of its ports to one another for fluid communication such that, in use, when the syringe is evacuated, the angio pump and the apparatus up to the catheter are charged with carbon dioxide and the catheter is inserted into the vascular system of a patient, the three way valve (i) can be manipulated to connect the syringe to the catheter for fluid communication such that withdrawal of the plunger of the syringe purges the catheter and (ii) thereafter can be manipulated to connect the catheter to the first hose for fluid communication such that evacuation of the angio pump injects carbon dioxide into said vascular system.
